(19) United States
(12) Patent Application Publication
(10) Pub. No.: US 2026/0259148 A1
(43) Pub. Date: Sep. 3, 2026

(54) METHOD AND SYSTEM FOR CHARACTERIZING A NON-METALLIC INCLUSION POPULATION

(71) Applicant: AKTIEBOLAGET SKF, GOTEBORG (SE)

(72) Inventors: Hanzheng HUANG, Houten (NL); Junbiao LAI, Houten (NL); Sebastien BLACHERE, Den Haag (NL)

(21) Appl. No.: 18/878,280

(22) PCT Filed: Jul. 6, 2022
(86) PCT No.: PCT/EP2022/068784

§ 371 Date: Dec. 23, 2024

(51) Int. Cl. G01N 21/94; G01M 13/04; G01N 1/28; G01N 1/32; G01N 21/95; G01N 21/956

(52) U.S. Cl. CPC G01N 21/94; G01M 13/04; G01N 1/286; G01N 1/32; G01N 21/951; G01N 21/95684; G01N 2001/2873; G01N 2201/126

(57) ABSTRACT

A method for characterizing a population of non-metallic inclusions in a component comprising a metal alloy includes cutting and polishing the component to provide an inspection plane, scanning at least one control area of the inspection plane with an optical microscope, using automated optical microscopy and image analysis software to automatically count members of the population of non-metallic inclusions in the at least one control area, determining at least one morphological parameter and a position of each member of the population of non-metallic inclusions within the at least one control area to produce measured data, and processing the measured data using an Extreme Value Analysis or a Generalized Pareto Distribution analysis technique to produce processed data characterizing the population of non-metallic inclusions in the finished or semi-finished component. The morphological parameter is an area, a length, a width and/or an aspect ratio of the non-metallic inclusions.

### TECHNICAL FIELD

The present invention concerns a method and system for characterizing a non-metallic inclusion population in a finished or semi-finished component comprising a metal alloy, such as steel. The present invention also concerns a method for modelling the fatigue life of a component, such as a bearing component, and a method for controlling the quality of a finished or semi-finished component, such as a bearing component.

### BACKGROUND OF THE INVENTION

Non-metallic inclusions are chemical compounds and non-metals that are present in metal alloys, such as steel. They are the product of chemical reactions, physical effects, and contamination and may be formed during the production of the metal alloy and during the manufacture of a component from the metal alloy.

Non-metallic inclusions that are present in components, such as bearing components, can act as stress raisers and may initiate the formation of cracks that propagate under stress reversals until a fatigue pit or spall is formed on the surface of the bearing component, which may cause eventual fatigue failure. Non-metallic inclusions can thereby shorten the useful service life of a component.

Given the importance of non-metallic inclusion content in relation to component fatigue performance, the characterization of inclusion populations in metal alloys and in components manufactured from metal alloys is of crucial importance. Several assessment methods for the analysis of non-metallic inclusions have consequently been developed.

Ultrasonic testing (UST) is a family of non-destructive testing techniques based on the propagation of ultrasonic waves in the component or material tested. In most common UST applications, very short ultrasonic pulse-waves with centre frequencies ranging from 0.1 to 15 MHz are transmitted into a component or material to detect internal flaws or to characterize the non-metallic inclusion population in the material. UST is however used only to quantify the number and size of macro-inclusions, i.e. non-metallic inclusions having a length greater than 200 μm) and not micro-inclusions, i.e., non-metallic inclusions that are formed as a result of a metal-alloy-making process and that have a length of 1 μm to 200 μm.

Microcomputed tomography (Micro-CT) is another non-destructive testing (NDT) method for the detection of material defects. Micro-CT is a 3D imaging technique utilizing X-rays to see inside a component or material, slice by slice. Micro-CT scanners capture a series of 2D planar X-ray images and reconstruct the data into 2D cross-sectional slices. These slices can be further processed into 3D models and printed as 3D physical objects for analysis. 3D micro-CT systems can be used to reveal a component's or material's internal features and to provide volumetric information about its microstructure. This technique can however only scan a very small volume of the test material, i.e., about 1 mm³. Such a volume is too small to characterize the non-metallic inclusion population in the stressed volume of bearing components.

Manual non-metallic inclusion counting and measuring is used to characterize the non-metallic inclusion population in raw materials in the form of bars, billets or plates, for the purpose of quality control. In manual non-metallic inclusion counting and measuring, test samples are taken from regions representing an average quality of bulk material.

A disadvantage of manual counting and measuring is that it does not provide useful information concerning the final products that are subsequently produced from the raw material being analyzed. The final products produced from the raw material will namely contain a different population of metallic inclusions than the raw material used to produce them. Such quality control of raw material by manual counting and measuring is therefore important and useful to raw material manufacturers, but not to a user of a component manufactured from that raw material.

Another disadvantage of manual counting and measuring is that it is time consuming and relies to a large extent on the operator's knowledge and experience and it is thereby prone to human error.

Due to the improvements that have been made in raw material production methods, such as in steelmaking technologies, metal alloys, such as steel, with the highest purity contain only a few large micro-inclusions and many unavoidable fine ones. Large micro-inclusions are however too rare to be directly measured in a practically small reference volume or area, so statistical distributions of large inclusions are often used to predict the unobserved extreme sizes in a large volume, such as in tons of steel or in thousands of bearings, by extrapolation on the basis of measurements on small test samples.

Extreme value theory, which concerns the statistical behaviour of extreme values in a single process, has been utilised to estimate the sizes of large inclusions in clean steels.

The international standard ASTM E2283 entitled ‘Standard Practice for Extreme Value Analysis (EVA) of Non-metallic Inclusions in Steel and Other Microstructural Features’ describes a methodology to statistically characterize the distribution of the largest indigenous non-metallic inclusions in steel specimens based upon quantitative metallographic measurements. This practice enables the experimenter to estimate the extreme value distribution of inclusions in steels.

The EVA technique is based on the Gumbel distribution of statistical extreme value theory. The extreme values in this approach come from block maxima (BM) sampling, in which multiple separate and equally sized samples are scanned and only the largest inclusion present in each of them are taken for analysis. A disadvantage of this technique is that only the largest inclusion is considered in this method while other large inclusions, which may also cause failure if they are present in the stressed volume of a component are ignored.

A disadvantage with the practice described in ASTM E2283 is that the non-metallic inclusions must be manually counted and measured, which is tedious and time consuming, and the large areas required in order to obtain representative statistical data for heterogeneous steels are rarely investigated.

### SUMMARY OF THE INVENTION

An object of the invention is to provide an improved method for characterizing a non-metallic inclusion population in component made of, or comprising a metal alloy, such as steel.

The method comprises the step of cutting and polishing a finished or semi-finished component to provide an inspection plane. The method is not therefore carried out on raw material, but a finished or partially finished component. The method comprises the step of scanning at least one control area that constitutes at least part of the inspection plane, and using automated optical microscopy and image analysis software to automatically count non-metallic inclusions within the at least one control area and measure or determine at least one morphological parameter, such as an area and/or a length and/or a width and/or an aspect ratio, and a position, i.e. location coordinates, of some or all of the non-metallic inclusions within the at least one control area to produce measured data. The method also comprises the step of processing the measured data using an Extreme Value Analysis (EVA) or a Generalized Pareto Distribution (GPD) analysis technique to characterize the non-metallic inclusion population in the finished or semi-finished component.

Generalized Pareto Distribution (GPD) analysis technique is an EVA technique that is based on the Generalised Pareto Distribution (GPD) of values larger than a specified threshold. GPD analysis considers not only the largest non-metallic inclusion in a control area (as in EVA analysis), but all non-metallic inclusions larger than a threshold value in the total inspection area. Using a Generalized Pareto Distribution (GPD) analysis technique thereby offers metallurgists a better representation of a non-metallic inclusion population than that given by the EVA analysis technique, since it is not always the largest non-metallic inclusion that is a fatigue initiator in a component. The second or third largest non-metallic inclusion etc. can also be a fatigue initiator if it is subjected to sufficient stress during the use of the component.

The EVA or GPD statistical parameters generated by the method according to the invention can be used as input data for a fatigue model to predict the durability or fatigue strength of a component under specified operating conditions.

The method according to the invention provides an efficient and reliable way of measuring and characterizing inclusions, i.e. both macro- and/or micro-inclusions. The method makes it possible to characterize a large number of non-metallic inclusions, such as several hundreds of thousands of non-metallic inclusions, and/or to analyze a large inspection area, such as an area of several thousands of square millimetres, in a relatively short time. Since a lot of measured data is considered, the accuracy of the characterization of the non-metallic inclusion population is increased compared known assessment methods. Additionally, errors that are often caused by manual measurement are eliminated.

Another advantage of the method is that since a region of a finished or semi-finished component is analyzed rather than a region of raw material representing the average quality of the bulk material, the characterization of the non-metallic inclusion population is more relevant and useful for component manufacturers and suppliers. Non-metallic inclusions produced during the process(es) used to manufacture the finished component the process(es) used to partially manufacture the semi-finished component will namely be analyzed. The EVA or GPD statistical parameters generated by the method according to the invention may therefore be provided to a component manufacturer or supplier so that improvements may be made to a component's manufacturing process, if necessary.

The term “cutting and polishing”, as used herein, is intended to mean any method for preparing a flat, defect-free surface that is suitable for examination using an automated optical microscope. “Cutting and polishing” may include at least one of the following metallurgical techniques: cutting, grinding, polishing, buffing, electropolishing, flattening, honing. Artefacts such as corrosion pits, dust particles and scratches must be eliminated in the specimen preparation.

The term “optical microscope”, as used herein, is intended to mean a microscope that uses visible light and a system of lenses to generate a magnified image of non-metallic inclusions. The word “motorized” is intended to mean that the light optical microscope is configured to be moveable and/or the microscope stage on which the finished or semi-finished component that is to be analyzed is placed is configured to be moveable so that the at least one control area can be scanned.

The term “length of a non-metallic inclusion”, as used herein, is intended to mean the measurement or extent of a non-metallic inclusion, as seen in a control area, from end to end.

The term “width of a non-metallic inclusion”, as used herein, is intended to mean the measurement of a non-metallic inclusion, as seen in a control area, taken at right angles to the length of the non-metallic inclusion.

The term “measure or determine” is intended to mean that one or more morphological parameters are measured directly or determined from an analysis of the at least one control area. For example, the area of a non-metallic inclusion may be determined using an equivalent circle diameter (ECD).

According to an embodiment of the invention the area of a non-metallic inclusion is measured or determined by the method according to the invention since area is the most accurate morphological parameter that can be measured or determined using image analysis software.

According to an embodiment of the invention the at least one control area has an area of at least 150 mm², or at least 200 mm², 300 mm², 400 mm², 500 mm², or up to at least 1000 mm² or more. A plurality of control areas of this size may namely be used if an EVA analysis technique is used.

According to an embodiment of the invention the method comprises the step of providing the inspection plane so that it at least partly extends through a volume of the finished or semi-finished component that is stressed during the use of the component. The non-metallic inclusion population in a stressed volume of the finished or semi-finished component will thereby by characterized, which is of great importance for a future application. The term “stressed volume”, as used herein, is intended to mean the volume slightly underneath a surface, such as a raceway surface in a bearing component, which is subjected to Hertzian contact stress or alternating Hertzian contact stress when a component is used. Maximum shear stress is namely generated slightly underneath such a surface when a component is in use and contact fatigue is most like to take place within this stressed volume. If stress raisers such as non-metallic inclusions are present in such a region of high stress, delamination may take place.

It should be noted that the method according to the present invention may be used to analyze a finished component before its use (to predict its useful service life in a future application), during its use (to determine remaining service life) and/or after its use (to investigate why a component may have failed). A “stressed volume” may therefore be a volume of a finished component that has been subjected to Hertzian contact stress or alternating Hertzian contact stress during its use.

According to an embodiment of the invention the finished or semi-finished component comprises a raceway surface and the method comprises the step of providing the inspection plane so that it at least partly extends parallel to the raceway surface and/or within 1 mm, or within 800 μm, or within 500 μm of the raceway surface.

The at least one control area preferably at least partly extends parallel to the raceway surface and/or within 1 mm, or within 800 μm, or within 500 μm of the raceway surface. Such an inspection plane orientation captures the non-metallic inclusions that are relevant for fatigue crack initiation and propagation.

It should be noted that the expression “parallel”, as used herein, is intended to mean parallel or substantially parallel.

According to an embodiment of the invention the method comprises the step of characterizing a micro-inclusion population, i.e., a population of non-metallic inclusions having a length from 1 μm to 10 μm or 1 μm to 20 μm, or 1 μm to 50 μm, 1 μm to 100 μm, or 1 μm up to and including 200 μm. A method according to the present invention may be used as a complement to a high-frequency ultrasonic technique that characterizes macro-inclusions.

According to an embodiment of the invention the finished or semi-finished component is one of the following: a bearing component, such as an inner or outer bearing ring, a bearing raceway, a roller bearing, a needle bearing, a tapered roller bearing, a spherical roller bearing, a toroidal roller bearing, a ball thrust bearing, a roller thrust bearing, a tapered roller thrust bearing, a wheel bearing, a hub bearing unit, a Compact Aligning Roller Bearing (CARB™), an angular contact ball bearing (ACBB), a deep groove ball bearing (DGBB), an angular contact ball bearing, a spherical roller bearing used in a continuous caster line, a backing bearing, a slewing bearing or a ball screw, a finished or semi-finished transmission component, such as a sprocket, a gear, a bushing, a hub, a coupling, a bolt, a screw, a shaft, such as a spindle shaft, a roller or roller mantle, a seal, a tool, a metal wheel, a wire, or any other finished or semi-finished component for an application in which it is subjected to Hertzian contact stress, or alternating Hertzian contact stress, or any finished or semi-finished structural component.

The term “structural component”, as used herein, is intended to mean any component that supports non-variable forces or weights (dead) and variable forces or weights (live loads), such as a column, girder, beam, support structure.

The finished or semi-finished component analyzed in the method according to the invention may comprise, or be made of steel, such as bearing steel, hardened steel, carbon steel, stainless steel, or any other metal alloy, such as a nickel-based superalloy, a titanium alloy, brass or bronze.

According to an embodiment of the invention the method comprises the step of recording the measured data. The measured data can thereby be processed at a later time and/or used for some other purpose at a later time. For example, the measured data may be used to determine the aspect ratio of non-metallic inclusions to classify the non-metallic inclusions since there is a correlation between the morphology and the chemical composition of non-metallic inclusions.

The present invention also concerns a system for automatically characterizing a non-metallic inclusion population in a finished or semi-finished component comprising a metal alloy, such as steel, bearing steel, hardened steel, carbon steel, stainless steel, or any other metal alloy, such as a nickel-based superalloy, a titanium alloy, brass or bronze.

The system comprises an automated optical microscope, such a motorized light optical microscope, and image analysis software configured to automatically count non-metallic inclusions within at least one control area and measure or determine at least one morphological parameter, such as an area and/or a length and/or a width and/or an aspect ratio, and a position of non-metallic inclusions within the at least one control area to produce measured data. The system also comprises a processor comprising a computer program that includes program code means executable by the processor to process the measured data using an Extreme Value Analysis (EVA) or a Generalized Pareto Distribution (GPD) analysis technique to characterize the non-metallic inclusion population in the finished or semi-finished component.

Optionally, the system may comprise a memory to store the measured data and/or any other data used by the system and/or generated by the system.

A system according to any of the embodiments described herein may be used to carry out a method according to any of the embodiments described herein.

The present invention further concerns a method for modelling the fatigue life of a component, such as a bearing component, The method comprises the step of using EVA or GPD statistical parameters obtained using a method according to any of the embodiments described herein and/or a system according to any of the embodiments described herein to predict the fatigue strength of a bearing component under specified operating conditions. The method and system according to the present invention may thereby be used to help manufacturers find ways to prolong the useful service life of a component, such as a bearing component.

Since the method according to the invention provides a large amount of measured data, the accuracy of a fatigue life model using such measured data will be improved.

The present invention further concerns a method for controlling the quality of a finished or semi-finished component, such as a bearing component. The method comprises the step of using EVA or GPD statistical parameters obtained using a method according to any of the embodiments described herein and/or a system according to any of the embodiments described herein. Such a quality control method may be used by manufacturers and suppliers to check the quality of a finished or semi-finished component.

### BRIEF DESCRIPTION OF THE DRAWINGS

The present invention will hereinafter be further explained by means of non-limiting examples with reference to the appended figures where;

FIG. 1 shows an example of a component comprising a population of non-metallic inclusions that can characterized using a method according to an embodiment of the invention,

FIGS. 2 & 3 show the extension of an inspection plane and/or a control area through a finished or semi-finished component,

FIG. 4 is an image representing a population of non-metallic inclusions as seen by an automated optical microscope,

FIG. 5 shows block maxima sampling using an Extreme Value Analysis (EVA) analysis technique,

FIG. 6 shows peak-over-threshold (POT) sampling using a Generalized Pareto Distribution (GPD),

FIG. 7 shows the probability of failure of a component determined using a method according to the present invention,

FIG. 8 is a flow chart showing the steps of a method according to according to an embodiment of the invention, and

FIG. 9 schematically shows a system according to an embodiment of the present invention.

It should be noted that the drawings have not necessarily been drawn to scale and that the dimensions of certain features may have been exaggerated for the sake of clarity.

It should also be noted that any feature described with reference to a particular embodiment of the method or system according to the invention in any part of this documents including the appended figures, may be used in any other embodiment of the method or system according to the invention unless the description explicitly excludes such a combination.

### DETAILED DESCRIPTION OF EMBODIMENTS

FIG. 1 schematically shows an example of a component 10 comprising a population of non-metallic inclusions that can be characterized using a method according to an embodiment of the invention, namely a rolling element bearing comprising an inner ring 12, an outer ring 14, a set of rolling elements 16, a cage (not shown) and raceway surfaces 18.

At least part of the component 10 or the whole component may be made from steel. For example the bearing rings 12, 14 may be made from 100Cr6, a steel containing approximately 1% carbon and 1.5% chromium. Typically, the same material is used for rolling elements 16 as for bearing rings 12, 14. When required, rolling elements 16 can however be made from ceramic material, whereby the component 10 is a hybrid bearing. A bearing surface 18 of the component may comprise a pure metal, such as iron, nickel, titanium, copper, aluminium, tin or zinc, or a metal alloy, such as steel, carbon steel, stainless steel, a nickel-based superalloy, a titanium alloy, brass or bronze.

A component 10, as described herein, may have a width or diameter up to a few metres in size and have a load-carrying capacity up to many thousands of tonnes. A component 10 may namely be of any size and have any load-carrying capacity. The component 10 may be used in industries such as metals, mining, mineral processing, cement, automotive, railway, renewable or traditional energy, pulp or paper, or marine.

The method according to the invention comprises the step of cutting and polishing a finished or semi-finished component 10 to provide an inspection plane 20.

The method comprises the step of scanning at least one control area 22 that constitutes at least part of the inspection plane 20 and using automated optical microscopy and image analysis software to automatically count non-metallic inclusions within the at least one control area 22 and measure or determine at least one morphological parameter, such as an area and/or a length and/or a width and/or an aspect ratio, and a position of non-metallic inclusions within the at least one control area 22.

The method according to the invention comprises the step of analyzing the population of non-metallic inclusions 24 present in the at least one control area 22 using one of two extreme value analysis techniques, i.e. an Extreme Value Analysis (EVA) or a Generalized Pareto Distribution (GPD) analysis technique, to characterize the non-metallic inclusion population in the finished or semi-finished component 10.

The chemical composition of the non-metallic inclusions is not determined in the method according to the invention. Non-metallic inclusions are classified according to their morphological similarity and not their chemical identity.

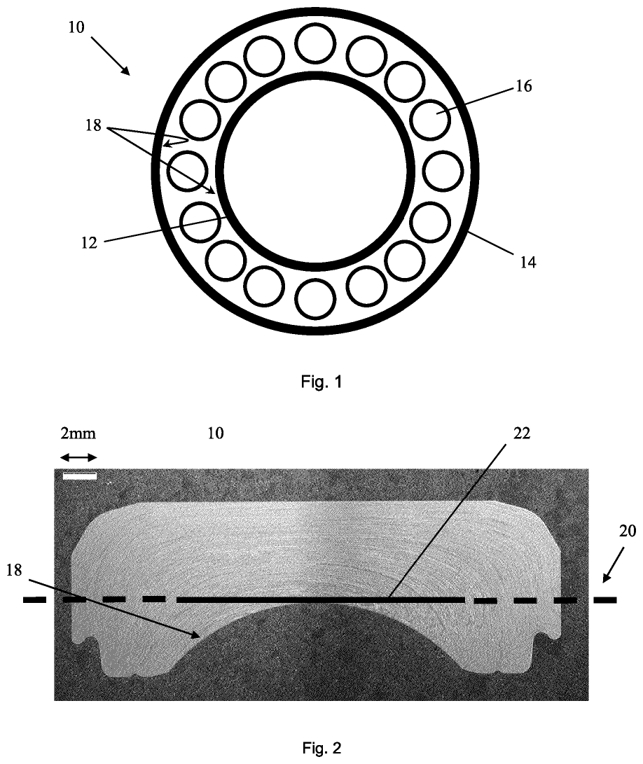

FIG. 2 shows a component 10 comprising a raceway surface 18. An inspection plane 20 is provided by cutting the component 10 along the plane indicated by the dotted line 20 below the raceway surface 18, and polishing the sectioned plane using a plurality of polishing steps whereby 200 μm of material is removed in each polishing step to produce an inspection plane 20 that at least partially extends within 1 mm of the raceway surface 18 and/or so that at least part of the inspection plane 20 is parallel to the raceway surface 18.

An automated optical microscope, such as a motorized light optical microscope, is used to scan one or more control areas 22 (indicated by a solid lane along the inspection plane 20 in FIG. 2) constituting at least a part of the inspection plane 20. Image analysis software is used to automatically count non-metallic inclusions within the scanned control area(s) 22 and measure or determine at least one morphological parameter, such as an area and/or a length and/or a width and/or an aspect ratio, and a position of non-metallic inclusions within the scanned control area(s) 22. The entire inspection plane 20 may be scanned by an automated optical microscope 28, whereby the whole inspection plane 20 constitutes a single control area 22 if a GPD analysis technique is used. Alternatively, the inspection plane 20 may be divided into a plurality of control areas 22 if an EVA analysis technique is used.

FIG. 3 also shows a component 10 comprising a surface 18 that is stressed during the use of the component 10. FIG. 3 shows which part of the component 10 will constitute at least one control area 22 once the component 10 has been cut and polished along a line parallel to the surface 18.

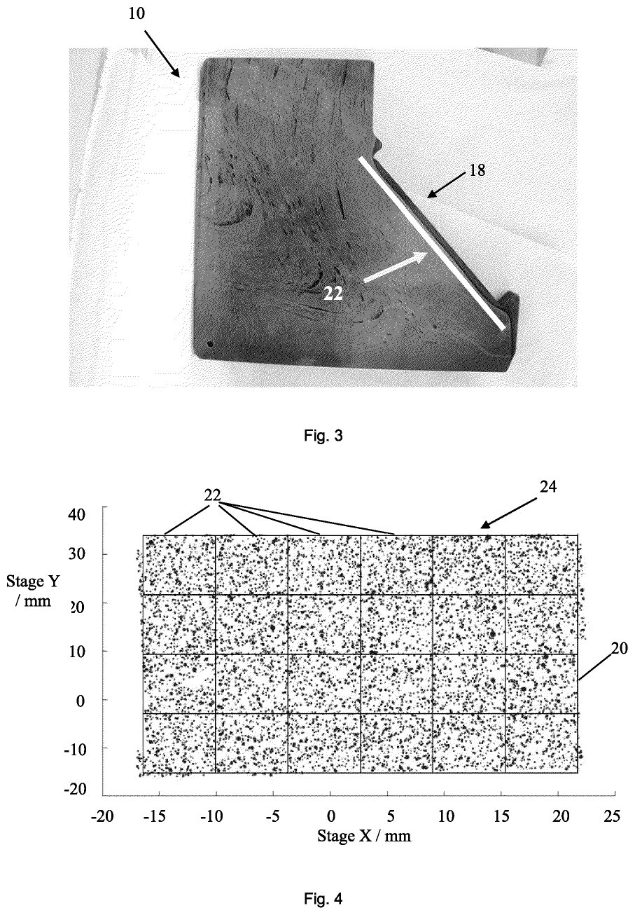

FIG. 4 is an image representing a population of non-metallic inclusions 24 as seen by an automated optical microscope. Image analysis is used to automatically count and measure at least one morphological parameter of the non-metallic inclusions in at least one control area 22.

According to an embodiment of the invention at least 100,000, or at least 200,000, or at least 500,000 non-metallic inclusions are analyzed.

FIG. 4 shows an inspection plane 20 divided into a plurality of control areas 22, namely twenty-four control areas. Twenty-four control areas is a default number of control areas since the EVA method described in the international standard ASTM E2283 requires 24 control areas of 150 mm², which ideally should be obtained from at least six different components to ensure sampling variability). Each of the control areas 22 are analyzed using an EVA analysis technique. An inspection plane 20 may however be divided into any number of control areas for EVA analysis of any suitable size in the method according to the invention. The largest non-metallic inclusion in each control area 22 is determined.

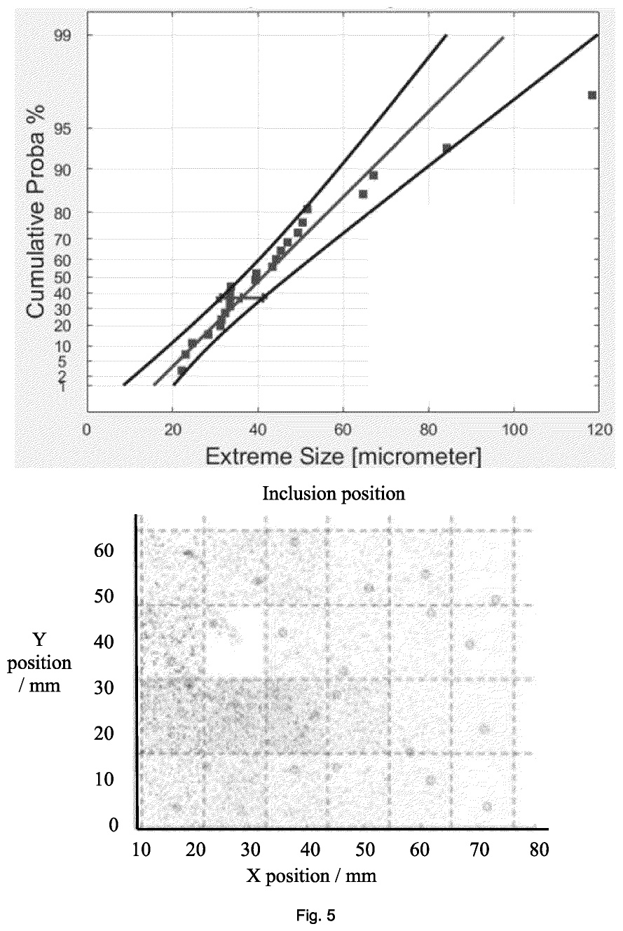

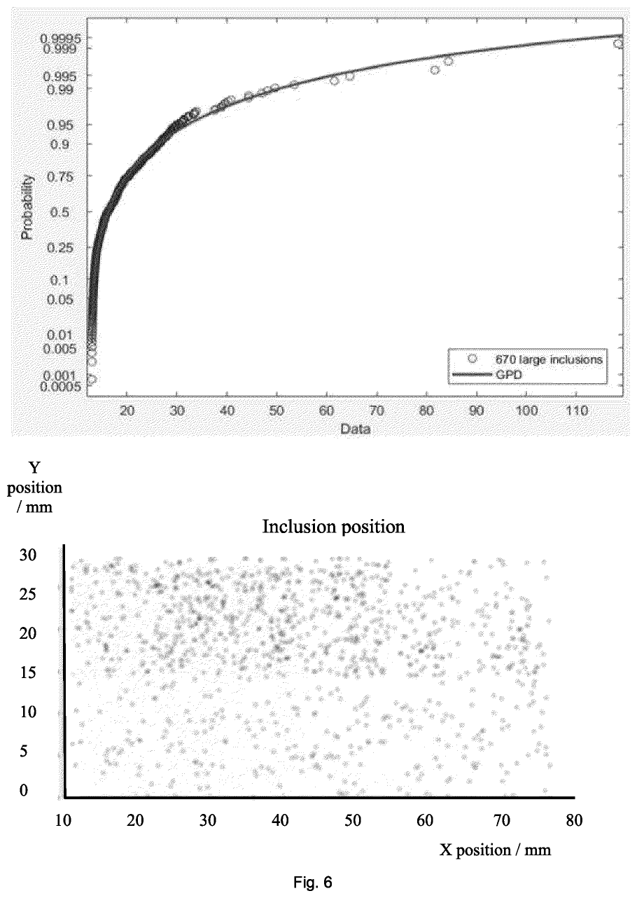

The differences on sampling and curve fitting of EVA and GPD are illustrated in FIGS. 5 and 6 using the same set of non-metallic inclusions.

FIG. 5 shows block maxima sampling using an EVA analysis technique using twenty-four maxima. FIG. 5 is namely a plot of the largest non-metallic inclusions that have been measured in each of a plurality of control areas 22.

FIG. 6 shows peak-over-threshold (POT) sampling using a Generalized Pareto Distribution (GPD).

A comparison of FIGS. 5 and 6 shows the advantage of using a lot more non-metallic inclusion sizes in the GPD technique over using a standard twenty-four maxima in an EVA technique.

An EVA plot is a reduced variant plot of a Gumbel cumulative distribution function (CDF):

$${F}_{{X}_{max}^{( N )}} ( x ) = exp \{ - exp ( - 1 ( \frac{x - λ}{δ} ) ) \}$$

$$y = - ln ( - ln ( {F}_{{X}_{max}}^{( N )} ( x ) ) ) = \frac{x - λ}{δ}$$

where X^{(N)}ₘₐₓ is the largest non-metallic inclusion size of each control volume, λ and δ are the location parameters and the scale parameter of the Gumbel distribution respectively.

The control volume V₀ is estimated by V₀=A₀×d, where A₀ is the control area size (150 mm² by default) and d is the mean of the measured sizes of the n maximum non-metallic inclusion inclusions.

Characteristic size is the non-metallic inclusion size corresponding to the fitted CDF

$${F}_{{X}_{max}}^{( N )} ( x ) = 99.9 \% ,$$

followed by ±95% CI, which is equal to ±2. SE(x).

SE(x) is the standard error.

The CDF of GPD, F (x), is written as:

$$F ( x ) = 1 - {( 1 - k \cdot \frac{x - θ}{σ} )}^{\frac{1}{k}}$$

where x is the excess of non-metallic inclusion sizes over the threshold θ, σ and k are the scale and shape parameters of the GPD.

The control volume V₀ is estimated by V₀=A_{ref}×d, where A_{ref} is the total scanned control area size and d is the mean of the measured sizes of the non-metallic inclusions larger than the threshold, θ.

Characteristic size is the non-metallic inclusion size corresponding to the fitted CDF

$$( 1 - \frac{{A}_{ref}}{N \cdot 1000 {A}_{0}} ) ,$$

where A₀ is control area size (150 mm² by default) and N is the total number of non-metallic inclusions larger than e.

The EVA or GPD analysis techniques thereby generate statistical parameters that may be used to characterize the population of non-metallic inclusions 24.

The values presented in the tables below are examples of EVA and GPD statistical parameters that were determined for different types of non-metallic inclusions in terms of their morphology.

EVA parameters

| | Single | | Elongated |
|---|---|---|---|
| | globular | Elongated | inclusions |
| | inclusions | inclusions | (Length) |
| | (ECD) [μm] | (ECD) [μm] | [μm] |
| λ [μm] | 14.89 | 14.1 | 37.66 |
| δ [μm] | 3.41 | 2.87 | 9.76 |
| V₀ [mm³] | 2.84 | 2.63 | 7.41 |
| Characteristic size [μm] | 38.46 ± 7.77 | 33.93 ± 6.53 | 105.05 ± 22.20 |

λ and δ are the location and scale parameters of the Gumbel distribution respectively.
V₀ is the control volume.

GPD parameters

| | Single globular | Elongated |
|---|---|---|
| | inclusions | inclusions |
| | (ECD) [μm] | (ECD) [μm] |
| k [μm] | 0.08 | −0.07 |
| σ [μm] | 3.01 | 3.68 |
| θ [μm] | 13 | 13 |
| V₀ [mm³] | 58.57 | 59.21 |
| Characteristic size [μm] | 48.45 | 34.92 |

σ and k are the scale and shape parameters of the GPD and θ is the threshold.
V₀ is the control volume.

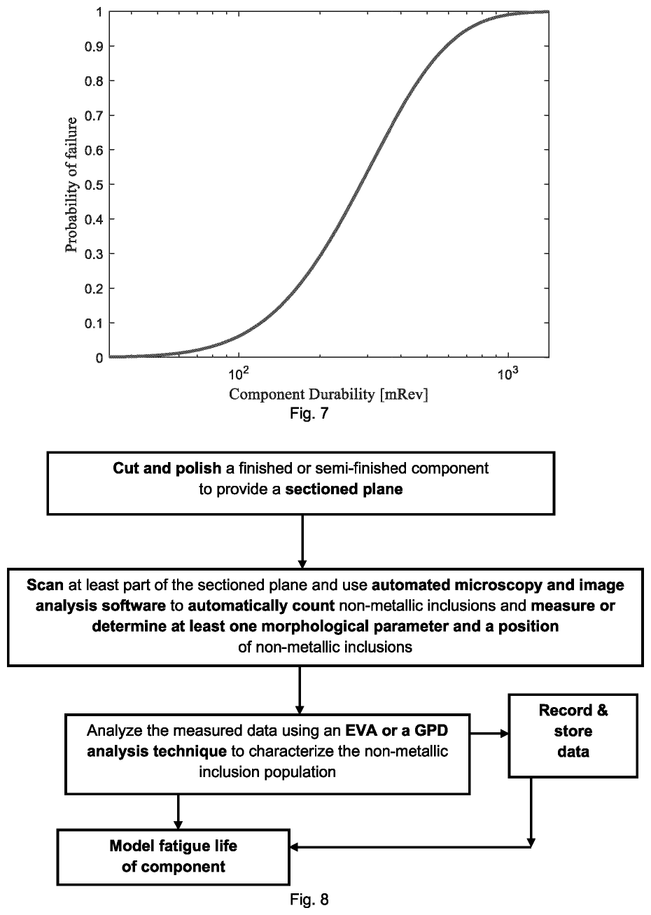

The EVA or GPD statistical parameters generated by the method according to the invention can be used as input for a fatigue model to predict the durability or fatigue strength of a component under specified operating conditions, as shown in FIG. 7. FIG. 7 namely shows the probability of failure of a component determined using a method according to the present invention. It provides a prediction of the performance of a randomly selected component of the type subjected to a method according to the invention.

FIG. 8 is a flow chart showing the steps of a method according to according to an embodiment of the invention. All of the automated microscopy, image analysis and data processing steps may be fully automated.

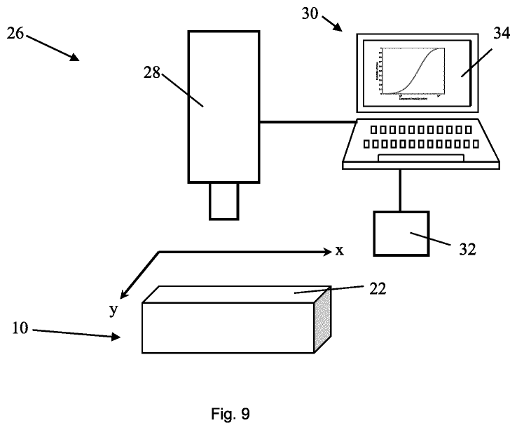

FIG. 9 schematically shows a system 26 for automatically characterizing a non-metallic inclusion population in a finished or semi-finished component 10 comprising a metal alloy according to an embodiment of the invention. The system 26 comprises an automated optical microscope 28, such as a motorized light optical microscope 28, and image analysis software configured to automatically measure or determine at least one morphological parameter, such as an area and/or a length and/or a width and/or an aspect ratio, and a position of non-metallic inclusions within a cut and polished inspection plane 22 of a finished or semi-finished component 10.

The automated optical microscope 28 may be a digital camera and/or a motorized stage. A control unit may be used to control the automated optical microscope 28 fully automatically. The system may be configured to save and reuse the automated optical microscope's setting parameters, to autofocus and acquire images, and optionally to record the results.

By properly adjusting the brightness, contrast and shade correction, non-metallic inclusions can be automatically detected as they appear as darker specks in a brighter metal alloy matrix, as shown in FIG. 4. The automated non-metallic inclusion counting and morphological parameter measuring may be performed at one fixed magnification, such as ×100 or ×200. The measured morphological parameters may be stored together with non-metallic inclusion location coordinates.

The system 26 may comprise a control unit (not shown) to control one of the following: the movement of the optical microscope 28 and/or a microscope stage on which the finished or semi-finished component 10, the lighting of the finished or semi-finished component 10, the magnification of the optical microscope 28. The non-metallic inclusions being viewed may namely be magnified by any suitable factor, such as 400x, 500x, 600x, 700x, 800x, 900x, 1000x or more.

The system 26 also comprises a processor 28 comprising a computer program that includes program code means executable by the processor to process the measured data using an Extreme Value Analysis (EVA) or a Generalized Pareto Distribution (GPD) analysis technique to characterize the non-metallic inclusion population in the finished or semi-finished component. The system 26 may also comprise a memory 32 to store the area and/or length and/or width and/or shape and/or position of non-metallic inclusions within a cut and polished inspection plane of a finished or semi-finished component or any other data used or generated by the system 26 and/or display means 34 to show data used or generated by the system 26.

Further modifications of the invention within the scope of the claims would be apparent to a skilled person.

1. A method for characterizing a population of non-metallic inclusions in a finished or semi-finished component comprising a metal alloy the method comprising:
cutting and polishing the finished or semi-finished component to provide an inspection plane,
scanning at least one control area of the inspection plane, with an optical microscope,
using automated optical microscopy and image analysis software to automatically count members of the population of non-metallic inclusions in the at least one control area,
determining at least one morphological parameter, and a position of each member of the population of non-metallic inclusions within the at least one control area to produce measured data, and processing the measured data using an Extreme Value Analysis or a Generalized Pareto Distribution analysis technique to produce processed data characterizing the population of non-metallic inclusions in the finished or semi-finished component,
wherein the at least one morphological parameter is selected from the group consisting of: an area, a length, a width and an aspect ratio of the non-metallic inclusions in the set of non-metallic inclusions.

2. The method according to claim 1, wherein a first control area of the at least one control area has an area of at least 150 mm².

3. The method according to claim 1, wherein providing the inspection plane comprises providing the inspection plane such that the inspection plane extends through a stressed volume of the finished or semi-finished component.

4. The method according to claim 1, wherein the population of non-metallic inclusions includes micro-inclusions.

5. The method according to claim 1, wherein the finished or semi-finished component is selected from the group consisting of: an inner or outer bearing ring, a bearing raceway, a roller bearing, a needle bearing, a tapered roller bearing, a spherical roller bearing, a toroidal roller bearing, a ball thrust bearing, a roller thrust bearing, a tapered roller thrust bearing, a wheel bearing, a hub bearing unit, a Compact Aligning Roller Bearing, an angular contact ball bearing, a deep groove ball bearing, an angular contact ball bearing, a spherical roller bearing configured for use in a continuous caster line, a backing bearing, a slewing bearing and a ball screw.

6. The method according to claim 1, wherein the finished or semi-finished component comprises a raceway surface and wherein the inspection plane at least partly extends in a direction parallel to the raceway surface and/or at least partly extends within 1 mm of the raceway surface.

7. The method according to claim 1 including recording the measured data and/or the processed data.

8. A system for automatically characterizing a population of non-metallic inclusions in a finished or semi-finished component comprising a metal alloy the system comprising:
an automated optical microscope configured to scan a cut and polished inspection plane of the finished or semi-finished component, and
a processor operatively connected to the automated optical microscope and configured to run image analysis software to automatically count members of the population of non-metallic inclusions within at least one control area of the inspection plane and to determine at least one morphological parameter and a position of each member of the population of non-metallic inclusions in the at least one control area to produce measured data,
the processor further being configured to process the measured data using an Extreme Value Analysis or a Generalized Pareto Distribution analysis technique to produce processed data a characterizing the population of non-metallic inclusions in the finished or semi-finished component.

9-10. (canceled)

11. The system according to claim 10,
including a memory operatively connected to the processor and configured to store the measured data and/or the processed data.

12. The system according to claim 10,
wherein the processor is further configured to predict a fatigue strength of the finished or semi-finished component under specified operating conditions using the processed data.

13. The system according to claim 12,
wherein the finished or semi-finished component is selected from the group consisting of: an inner or outer bearing ring, a bearing raceway, a roller bearing, a needle bearing, a tapered roller bearing, a spherical roller bearing, a toroidal roller bearing, a ball thrust bearing, a roller thrust bearing, a tapered roller thrust bearing, a wheel bearing, a hub bearing unit, a Compact Aligning Roller Bearing, an angular contact ball bearing, a deep groove ball bearing, an angular contact ball bearing, a spherical roller bearing configured for use in a continuous caster line, a backing bearing, a slewing bearing and a ball screw.

14. The system according to claim 12,
wherein the finished or semi-finished component is selected from the group consisting of: a sprocket, a gear, a bushing, a hub, a coupling, a bolt, a screw, a shaft, a roller or roller mantle, a seal, a tool, a metal wheel, and a wire.

15. The method according to claim 1,
further including predicting a fatigue strength of the finished or semi-finished component under specified operating conditions using the processed data.

16. The method according to claim 15,
wherein the finished or semi-finished component is selected from the group consisting of: an inner or outer bearing ring, a bearing raceway, a roller bearing, a needle bearing, a tapered roller bearing, a spherical roller bearing, a toroidal roller bearing, a ball thrust bearing, a roller thrust bearing, a tapered roller thrust bearing, a wheel bearing, a hub bearing unit, a Compact Aligning Roller Bearing, an angular contact ball bearing, a deep groove ball bearing, an angular contact ball bearing, a spherical roller bearing configured for use in a continuous caster line, a backing bearing, a slewing bearing and a ball screw.

17. The method according to claim 15,
wherein the finished or semi-finished component is selected from the group consisting of: a sprocket, a gear, a bushing, a hub, a coupling, a bolt, a screw, a shaft, a roller or roller mantle, a seal, a tool, a metal wheel, and a wire.

18. The method according to claim 1,
wherein the finished or semi-finished component is selected from the group consisting of: a sprocket, a gear, a bushing, a hub, a coupling, a bolt, a screw, a shaft, a roller or roller mantle, a seal, a tool, a metal wheel, and a wire.
